(19) United States
(12) Patent Application Publication
(10) Pub. No.: US 2008/0220437 A1
(43) Pub. Date: Sep. 11, 2008

(54) Substrate Material for Handling and Analysing Samples

(75) Inventors: Dirk Jan Broer, Geldrop (NL); Roel Penterman, Eindhoven (NL); Emiel Peeters, Eindhoven (NL); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/064,652

(22) PCT Filed: Aug. 17, 2006
(86) PCT No.: PCT/IB2006/052842

§ 371 Date: Feb. 25, 2008

(30) Foreign Application Priority Data
Aug. 26, 2005 (EP) 05107853.3

(51) Int. Cl. C09K 19/12; G01N 33/483

(52) U.S. Cl. 435/6; 252/299.65; 435/287.2; 435/288.4

(57) ABSTRACT

The invention relates to a substrate material for analyzing one or more fluid samples for the presence, amount or identity of one or more analytes in the samples, whereby the substrate material is adapted in that way that a flow of the sample or parts thereof in and/or with the substrate material is influenced and/or caused by phase transitions, preferably temperature-inducible phase transitions, in selected areas of the substrate material.

The present invention is directed to the field of devices for the handling and/or detection of one or more analytes in a sample, especially to the field of devices for handling and the detection of biomolecules in solution.

The present invention is directed to the handling and the detection of analytes in fluids, especially to the detection of biomolecules in solution. The detection usually occurs in that way, that the fluid to be analyzed is provided on a substrate material, which contains binding substances for the analytes which are subject of the detection. Such a capture probe may be a corresponding DNA-strand in case the analyte is also a DNA-Strand. The analytes in the fluid, which are usually equipped with a label, preferably an optical fluorescence label, will then be captured by the binding substance (in case of two complementary DNA strands this process is called hybridization) and remain there even after the fluid is removed. The analyte may then be detected.

However, usually the fluid is simply brought on the sample without any possibility to control the flow inside the substrate material. Such a flow may be controlled by microfluidic technology, but this requires a sophisticated layout of the analysis device and cannot be applied in all applications.

It is therefore an object of the present invention to provide a device and/or a substrate material for a device, which allows a quicker detection with a minute amount of analyte that needs to be present in the fluid.

This object is solved by a substrate material according to claim 1 of the present invention. Accordingly, a substrate material for use in a device analyzing one or more samples for the presence, amount or identity of one or more analytes in the samples is provided, whereby the substrate material is adapted in that way that a flow of the sample or parts thereof in and/or with the substrate material is influenced and/or caused by phase transitions in selected areas of the substrate material.

By doing so, a lesser amount of sample is needed and a sophisticated microfluidic layout of the device can be avoided.

The term “sample” according to the present invention includes fluid samples as well as solid samples which dissolve when being provided with the substrate material.

In the sense of the present invention, the term “flow of fluid or parts thereof in and/or with the substrate material” includes especially one or more of the following features:
According to one preferred embodiment of the present invention, the flow of the whole fluid in and/or with the substrate material is influenced and/or caused by phase transitions in selected areas of the substrate material;
According to one preferred embodiment of the present invention, the flow of parts of the fluid in and/or with the substrate material is influenced and/or caused by phase transitions in selected areas of the substrate material, preferably of particles or larger biomolecules in the fluid, as will be described later on;
According to one preferred embodiment, the flow of the whole fluid in the substrate material is influenced and/or caused by phase transitions in selected areas of the substrate material; for this reason, the substrate material is e.g. porous or allows a certain solubility of the fluid within the substrate material, as will be described later on;
According to one preferred embodiment, the flow of the whole fluid with the substrate material is influenced and/or caused by phase transitions in selected areas of the substrate material; for this reason, the phase transition in the substrate material influences or controls also e.g. further layers or other means with are allocated or provided with the substrate material.

In the sense of the present invention, the term “phase transition” means especially the transition from an ordered state to a less ordered state or an inverse transition form an less ordered to an ordered state.

A—non limiting—example for a phase transition that is especially meant with the present invention is the transition from the crystalline state to the amorphous isotropic state. It is known that the solubility of species is much higher in the amorphous state of matter than in the crystalline state. Further—non limiting—examples for a phase transition that is especially meant with the present invention are also the phase transitions relating liquid crystalline materials such as the transition from the nematic state to the isotropic state, the transition from the smectic state to the isotropic state or to the nematic state, further smectic phase transitions, for instance from the smectic B state to the smectic A state, where also a decrease in order is involved, transitions in solubility might occur.

In the sense of the present invention, the term “selected areas” may also include the substrate material as a whole. However usually it is preferred that only a part of the substrate material is subjected to a phase transition in order to influence and/or cause a flow of fluid in and/or with the sample.

According to a preferred embodiment of the present invention, the substrate material is adapted in that way that a directed flow of fluid or parts thereof in and/or with the substrate material is influenced and/or caused by phase transitions in selected areas of the substrate material. The term “directed” means especially that the direction, strength, lateral dispersion and/or distribution of the flow of fluid or parts thereof in and/or with the substrate material is influenced and/or caused by phase transitions in selected areas of the substrate material.

According to a preferred embodiment of the present invention, the phase transitions in the substrate material include reversible phase transitions.

By doing so, the flow in and/or with the substrate material may be controlled easier and more precise. Furthermore it is also possible to re-use the substrate material.

According to a preferred embodiment of the present invention, the substrate material is adapted in that way the interaction of the fluid to and/or in the substrate material changes when a phase transfer occurs.

By interaction is especially meant and/or included
the solubility of the fluid or parts of the fluid within the substrate material
the dispersibility of the fluid within the substrate material
the dispersibility of analyte particles within the substrate material. In this regard, it is taken reference to the following paper: ‘Drag on particles in a nematic suspension by a moving nematic-isotropic interface’ by J L West et al. Physical Review E, 2002, which is hereby incorporated by reference
the adhesion of the fluid or analyte particles to the substrate material The term “dispersibility” in the sense of the present invention means or includes especially the ability to disperse the fluid in the substrate material and/or the ability to mix or to blend other than by dissolving.

According to a preferred embodiment of the present invention, the substrate material is adapted in that way that solubility and/or dispersibility and/or adhesion of the fluid or parts of the fluid to and/or in the substrate material changes when a phase transfer occurs.

By doing so, a flow of fluid in and/or with the substrate material can be caused and/or influenced quite easily. Preferably, the phase transition changes the substrate material from a state, where the solubility and/or dispersibility and/or adhesion of the fluid to and/or in the substrate material is high to a state, where the solubility and/or dispersibility and/or adhesion of the fluid to and/or in the substrate material is low. Then the fluid will flow from the area(s) of the substrate material, where this phase transition had occurred to different areas, which still are in the initial state. (or in the opposite/different phase).

According to a preferred embodiment of the present invention, the substrate material is adapted in that way that a flow of macroparticles in the fluid in and/or with the substrate material is influenced and/or caused by phase transitions in selected areas of the substrate material. The term “macroparticles” in the fluid means especially larger biomolecules such as DNA-strands, peptides, enzymes, antibodies, biomarkers, and proteins. By doing so, an analysis of these particles in the fluid can be achieved more easily and effectively.

According to a preferred embodiment of the present invention, the substrate material is adapted in that way that a flow of macroparticles in the fluid in and/or with the substrate material is influenced and/or caused by phase transitions in selected areas of the substrate material, whereby the macroparticles have an average diameter and/or average dimension of ≧1 nm and ≦50 μm. These ranges have proven in practice to be best suitable within the present invention. Preferably, the macroparticles have an average diameter of ≧2 nm and ≦5 μm, more preferred macroparticles ≧5 nm and ≦1 μm and most preferred ≧10 nm and ≦0.1 μm.

According to a preferred embodiment of the present invention, the substrate material is adapted in that way that a flow of macroparticles in the fluid in and/or with the substrate material is influenced and/or caused by a solubility transition of the macroparticles upon the phase transition. By doing so, this solubility transition can be one of the driving forces for transport of the macroparticles from one location, being the location that has the state of the lowest solubility, to another location where the solubility is higher.

According to a preferred embodiment of the present invention, the substrate material is adapted in that way that a flow of macroparticles in the fluid in and/or with the substrate material is influenced and/or caused by a change in dispersibility of macroparticles. It has been found that in some applications, especially crystalline and liquid-crystalline materials show a tendency to transport particles to their domain boundaries. In the case of crystals it is the lattice energy of the crystal that expels material that does not fit in the crystal lattice to its boundary. In the case of liquid crystals the elastic energy of the liquid crystal when its alignment is disturbed by the presence of the macroparticle is the driving force for this behavior of expulsion.

According to another preferred embodiment of the present invention, the substrate material is adapted in that way that a size-selective or size-dependent flow of macroparticles in the fluid in and/or with the substrate material is influenced and/or caused by phase transitions in selected areas of the substrate material.

According to a preferred embodiment of the present invention, the substrate material is adapted in that way that more than one phase transition is possible with the substrate material. By doing so, a more controlled and in most applications even selective flow of the sample or parts thereof in and/or with the substrate material is feasible. Such a substrate material may be e.g. a liquid crystal material (as will be described later on), in which a phase transition from a nematic to a smectic as well as a phase transition from a nematic to an isotropic state is possible. Some particles or components of the sample may be caused only to flow when the phase transition from nematic to isotropic occurs, whereas others may also be forced to flow by a phase transition from the nematic to the smectic state or vice versa. By using these differences, a more differentiated flow of different components of the fluid is possible, thus resulting in a device which is capable of a higher resolution and velocity in analyzing the fluid.

According to a preferred embodiment of the present invention, the substrate material forms a layer with a thickness of ≧0.1 μm and ≦100 μm, preferably between ≧0.5 μm and ≦20 μm and most preferred between ≧1 μm and ≦10 μm. This has shown to be suitable in practice.

According to a preferred embodiment of the present invention, the phase transitions in the substrate material include temperature-inducible phase transitions. This allows a better control and monitoring of the selected areas of the substrate material simply by using heating means, e.g. heating plates and/or cooling means, e.g. Peltier-elements, which can be addressed quite precisely.

According to a preferred embodiment of the present invention, the phase transitions in the substrate material include temperature-inducible phase transitions with a transition temperature between ≧0° C. and ≦150° C., preferably between ≧20° C. and ≦120° C., more preferably between ≧30° C. and ≦100° C., and most preferred between ≧40° C. and ≦55° C. It has been shown in practice that these temperatures are most suitable.

According to a preferred embodiment of the present invention, the phase substrate material includes a liquid crystal material. By using such a material, the phase-transition between the smectic, nematic and/or isotropic state may be used to control the flow of the fluid.

In some applications of the present invention, in the case of the liquid crystalline transition often the transition is thermo-reversible, i.e. upon heating and upon cooling the transition occurs at about the same temperature. In the case of the crystalline transition melting upon heating usually occurs at a higher temperature than crystallization during cooling. This is because the nucleation of the crystallization retards the crystallization process and the phenomenon is known as supercooling where an isotropic liquid or liquid crystalline phase remains for a while in its non-equilibrium thermodynamic state. Improvement of the thermo reversibility can be enforced by the addition of nucleation agents.

A liquid crystal material in the sense of the present invention means especially an organic liquid material whose physical properties resemble those of a crystal in the formation of loosely ordered molecular arrays similar to a regular crystalline lattice and the anisotropic refraction of light. Different degrees of order are possible. The less ordered liquid crystal state is the nematic state. Here all molecules on the average are oriented into a similar direction, but there is no order in their centers of gravity. Higher ordered state are given by the so-called smectic phases in which the molecules on the average have a directional order and a positional order. Typically the molecules are ordered within layers. Depending on the degree of organization of the molecules within the layers one recognized a distinction in smectic phases denoted by a capital letter. For instance in the smectic A the molecules are ordered in layers with their average orientation perpendicular to the layer surface. Within the layer there is no positional order. In the smectic B phase the molecules are positioned hexagonally in the layers. The smectic C state resembles the state of order of smectic A, but the molecules are aligned under an angle with the layer surface. In the smectic D state the molecules are packed on a cubic lattice, etc. This all is common knowledge for those who are skilled in the field. In the case of polymeric liquid crystals the material exhibit the same types of order in their molecular parts, but the viscosity has become much higher such that in the liquid crystalline state they behave like pastes or elastomers in the case of crosslinked polymer systems.

By using a liquid crystal material, a control of the fluid in and/or with the substrate material can be achieved easily and effectfully. Without being determined, it is believed that one of the following mechanisms is responsible at least to a great deal to this effective control:

Liquid crystals tend to expel ‘foreign’ species driven by strong intermolecular interactions and the related elastic constants of the liquid crystal. If for instance foreign molecules (e.g. a fluid containing particles) are added to a liquid crystal system that is e.g. heated to a temperature where it is in its isotropic state, the particles distribute homogeneously. As soon as the temperature is lowered such that the material undergoes its phase transition e.g. to the nematic liquid crystalline state, the suspended particles are driven to concentrate themselves in the still isotropic areas.

It should be noted that the above described mechanism describes a phase transition from the isotropic state to the nematic state. It goes without saying that also further phase transitions, which can be effected in liquid crystal materials, e.g. from the smectic state to the nematic state may be employed as well.

According to a preferred embodiment of the present invention, the substrate material includes an aligned liquid crystal material. An alignment of the liquid crystals has shown for some applications to be beneficial to obtain more control over the directional flow, especially in case the detection of the analytes is optical based.

The term “alignment” in the sense of the present invention means especially that the liquid crystals exhibit long range orientation order. On average the long axis of the liquid crystalline molecules are oriented approximately parallel in a preferred direction. Optionally, the liquid crystals exhibit translation order as well.

According to a preferred embodiment of the present invention, the substrate material comprises a material according to structure I:

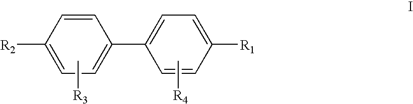

wherein R1, R2, R3 and/or R4 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

Generic group definition: Throughout the description and claims generic groups have been used, for example alkyl, alkoxy, aryl. Unless otherwise specified the following are preferred groups that may be applied to generic groups found within compounds disclosed herein:

alkyl: linear and branched C1-C8-alkyl,

long-chain alkyl: linear and branched C5-C20 alkyl

alkenyl: C2-C6-alkenyl, cycloalkyl: C3-C8-cycloalkyl,

alkoxy: C1-C6-alkoxy,

long-chain alkoxy: linear and branched C5-C20 alkoxy alkylene: selected from the group consisting of:

methylene; 1,1-ethylene; 1,2-ethylene; 1,1-propylidene; 1,2-propylene; 1,3-propylene; 2,2-propylidene; butan-2-ol-1,4-diyl; propan-2-ol-1,3-diyl; 1,4-butylene; cyclohexane-1,1-diyl; cyclohexan-1,2-diyl; cyclohexan-1,3-diyl; cyclohexan-1,4-diyl; cyclopentane-1,1-diyl; cyclopentan-1,2-diyl; and cyclopentan-1,3-diyl,

aryl: selected from homoaromatic compounds having a molecular weight under 300,

arylene: selected from the group consisting of: 1,2-phenylene; 1,3-phenylene; 1,4-phenylene; 1,2-naphtalenylene; 1,3-naphtalenylene; 1,4-naphtalenylene; 2,3-naphtalenylene; 1-hydroxy-2,3-phenylene; 1-hydroxy-2,4-phenylene; 1-hydroxy-2,5-phenylene; and 1-hydroxy-2,6-phenylene,

heteroaryl: selected from the group consisting of: pyridinyl; pyrimidinyl; pyrazinyl; triazolyl; pyridazinyl; 1,3,5-triazinyl; quinolinyl; isoquinolinyl; quinoxalinyl; imidazolyl; pyrazolyl; benzimidazolyl; thiazolyl; oxazolidinyl; pyrrolyl; carbazolyl; indolyl; and isoindolyl, wherein the heteroaryl may be connected to the compound via any atom in the ring of the selected heteroaryl,

heteroarylene: selected from the group consisting of: pyridindiyl; quinolindiyl; pyrazodiyl; pyrazoldiyl; triazolediyl; pyrazindiyl; and imidazolediyl, wherein the heteroarylene acts as a bridge in the compound via any atom in the ring of the selected heteroarylene, more specifically preferred are: pyridin-2,3-diyl; pyridin-2,4-diyl; pyridin-2,5-diyl; pyridin-2,6-diyl; pyridin-3,4-diyl; pyridin-3,5-diyl; quinolin-2,3-diyl; quinolin-2,4-diyl; quinolin-2,8-diyl; isoquinolin-1,3-diyl; isoquinolin-1,4-diyl; pyrazol-1,3-diyl; pyrazol-3,5-diyl; triazole-3,5-diyl; triazole-1,3-diyl; pyrazin-2,5-diyl; and imidazole-2,4-diyl, a —C1-C6-heterocycloalkyl, wherein the heterocycloalkyl of the —C1-C6-heterocycloalkyl is, selected from the group consisting of: piperidinyl; piperidine; 1,4-piperazine, tetrahydrothiophene; tetrahydrofuran; 1,4,7-triazacyclononane; 1,4,8,11-tetraazacyclotetradecane; 1,4,7,10,13-pentaazacyclopentadecane; 1,4-diaza-7-thia-cyclononane; 1,4-diaza-7-oxa-cyclononane; 1,4,7,10-tetraazacyclododecane; 1,4-dioxane; 1,4,7-trithia-cyclononane; pyrrolidine; and tetrahydropyran, wherein the heterocycloalkyl may be connected to the —C1-C6-alkyl via any atom in the ring of the selected heterocycloalkyl,

heterocycloalkylene: selected from the group consisting of: piperidin-1,2-ylene; piperidin-2,6-ylene; piperidin-4,4-ylidene; 1,4-piperazin-1,4-ylene; 1,4-piperazin-2,3-ylene; 1,4-piperazin-2,5-ylene; 1,4-piperazin-2,6-ylene; 1,4-piperazin-1,2-ylene; 1,4-piperazin-1,3-ylene; 1,4-piperazin-1,4-ylene; tetrahydrothiophen-2,5-ylene; tetrahydrothiophen-3,4-ylene; tetrahydrothiophen-2,3-ylene; tetrahydrofuran-2,5-ylene; tetrahydrofuran-3,4-ylene; tetrahydrofuran-2,3-ylene; pyrrolidin-2,5-ylene; pyrrolidin-3,4-ylene; pyrrolidin-2,3-ylene; pyrrolidin-1,2-ylene; pyrrolidin-1,3-ylene; pyrrolidin-2,2-ylidene; 1,4,7-triazacyclonon-1,4-ylene; 1,4,7-triazacyclonon-2,3-ylene; 1,4,7-triazacyclonon-2,9-ylene; 1,4,7-triazacyclonon-3,8-ylene; 1,4,7-triazacyclonon-2,2-ylidene; 1,4,8,11-tetraazacyclotetradec-1,4-ylene; 1,4,8,11-tetraazacyclotetradec-1,8-ylene; 1,4,8,11-tetraazacyclotetradec-2,3-ylene; 1,4,8,11-tetraazacyclotetradec-2,5-ylene; 1,4,8,11-tetraazacyclotetradec-1,2-ylene; 1,4,8,11-tetraazacyclotetradec-2,2-ylidene; 1,4,7,10-tetraazacyclododec-1,4-ylene; 1,4,7,10-tetraazacyclododec-1,7-ylene; 1,4,7,10-tetraazacyclododec-1,2-ylene; 1,4,7,10-tetraazacyclododec-2,3-ylene; 1,4,7,10-tetraazacyclododec-2,2-ylidene; 1,4,7,10,13 pentaazacyclopentadec-1,4-ylene; 1,4,7,10,13-pentaazacyclopentadec-1,7-ylene; 1,4,7,10,13-pentaazacyclopentadec-2,3-ylene; 1,4,7,10,13-pentaazacyclopentadec-1,2-ylene; 1,4,7,10,13-pentaazacyclopentadec-2,2-ylidene; 1,4-diaza-7-thia-cyclonon-1,4-ylene; 1,4-diaza-7-thia-cyclonon-1,2-ylene; 1,4-diaza-7thia-cyclonon-2,3-ylene; 1,4-diaza-7-thia-cyclonon-6,8-ylene; 1,4-diaza-7-thia-cyclonon-2,2-ylidene; 1,4-diaza-7-oxacyclonon-1,4-ylene; 1,4-diaza-7-oxa-cyclonon-1,2-ylene; 1,4diaza-7-oxa-cyclonon-2,3-ylene; 1,4-diaza-7-oxa-cyclonon-6,8-ylene; 1,4-diaza-7-oxa-cyclonon-2,2-ylidene; 1,4-dioxan-2,3-ylene; 1,4-dioxan-2,6-ylene; 1,4-dioxan-2,2-ylidene; tetrahydropyran-2,3-ylene; tetrahydropyran-2,6-ylene; tetrahydropyran-2,5-ylene; tetrahydropyran-2,2-ylidene; 1,4,7-trithia-cyclonon-2,3-ylene; 1,4,7-trithia-cyclonon-2,9-ylene; and 1,4,7-trithia-cyclonon-2,2-ylidene,

heterocycloalkyl: selected from the group consisting of: pyrrolinyl; pyrrolidinyl; morpholinyl; piperidinyl; piperazinyl; hexamethylene imine; 1,4-piperazinyl; tetrahydrothiophenyl; tetrahydrofuranyl; 1,4,7-triazacyclononanyl; 1,4,8,11-tetraazacyclotetradecanyl; 1,4,7,10,13-pentaazacyclopentadecanyl; 1,4-diaza-7-thiacyclononanyl; 1,4-diaza-7-oxa-cyclononanyl; 1,4,7,10-tetraazacyclododecanyl; 1,4-dioxanyl; 1,4,7-trithiacyclononanyl; tetrahydropyranyl; and oxazolidinyl, wherein the heterocycloalkyl may be connected to the compound via any atom in the ring of the selected heterocycloalkyl,

amine: the group —N(R)2 wherein each R is independently selected from: hydrogen; C1-C6-alkyl; C1-C6-alkyl-C6H5; and phenyl, wherein when both R are C1-C6-alkyl both R together may form an —NC3 to an —NC5 heterocyclic ring with any remaining alkyl chain forming an alkyl substituent to the heterocyclic ring,

halogen: selected from the group consisting of: F; Cl; Br and I,

pseudohalogen: selected from the group consisting of —CN, —SCN, —OCN, N3, —CNO, —SeCN

sulphonate: the group —S(O)2OR, wherein R is selected from: hydrogen; C1-C6-alkyl; phenyl; C1-C6-alkyl-C6H5; Li; Na; K; Cs; Mg; and Ca,

sulphate: the group —OS(O)2OR, wherein R is selected from: hydrogen; C1-C6-alkyl; phenyl; C1-C6-alkyl-C6H5; Li; Na; K; Cs; Mg; and Ca,

sulphone: the group —S(O)2R, wherein R is selected from: hydrogen; C1-C6-alkyl; phenyl; C1-C6-alkyl-C6H5 and amine (to give sulphonamide) selected from the group: —NR′2, wherein each R′ is independently selected from: hydrogen; C1-C6-alkyl; C1C6-alkyl-C6H5; and phenyl, wherein when both R′ are C1-C6-alkyl both R′ together may form an —NC3 to an —NCS heterocyclic ring with any remaining alkyl chain forming an alkyl substituent to the heterocyclic ring,

carboxylate derivative: the group —C(O)OR, wherein R is selected from: hydrogen; C1-C6-alkyl; phenyl; C1-C6-alkyl-C6H5; Li; Na; K; Cs; Mg; and Ca,

carbonyl derivative: the group —C(O)R, wherein R is selected from: hydrogen; C1-C6-alkyl; phenyl; C1-C6-alkyl-C6H5 and amine (to give amide) selected

from the group: —NR′2, wherein each R′ is independently selected from: hydrogen; C1-C6-alkyl; C1-C6-alkyl-C6H5; and phenyl, wherein when both R′ are C1-C6-alkyl both R′ together may form an —NC3 to an —NC5 heterocyclic ring with any remaining alkyl chain forming an alkyl substituent to the heterocyclic ring,

phosphonate: the group —P(O)(OR)2, wherein each R is independently selected from: hydrogen; C1-C6-alkyl; phenyl; C1-C6-alkyl-C6H5; Li; Na; K; Cs; Mg; and Ca,

phosphate: the group —OP(O)(OR)2, wherein each R is independently selected from: hydrogen; C1-C6-alkyl; phenyl; C1-C6-alkyl-C6H5; Li; Na; K; Cs; Mg; and Ca,

phosphine: the group —P(R)2, wherein each R is independently selected from: hydrogen; C1-C6-alkyl; phenyl; and C1-C6-alkyl-C6H5,

phosphine oxide: the group —P(O)R2, wherein R is independently selected from: hydrogen; C1-C6-alkyl; phenyl; and C1-C6-alkyl-C6H5; and amine (to give phosphonamidate) selected from the group: —NR′2, wherein each R′ is independently selected from: hydrogen; C1-C6-alkyl; C1-C6-alkyl-C6H5; and phenyl, wherein when both R′ are C1-C6-alkyl both R′ together may form an —NC3 to an —NC5 heterocyclic ring with any remaining alkyl chain forming an alkyl substituent to the heterocyclic ring.

polyether: chosen from the group comprising —(O—CH₂—CH(R))ₙ—OH and —(O—CH₂—CH(R))ₙ—H whereby R is independently selected from: hydrogen, alkyl, aryl, halogen and n is from 1 to 250.

Unless otherwise specified the following are more preferred group restrictions that may be applied to groups found within compounds disclosed herein:

alkyl: linear and branched C1-C6-alkyl,

long-chain alkyl: linear and branched C5-C10 alkyl, preferably linear C6-C8 alkyl

alkenyl: C3-C6-alkenyl,

cycloalkyl: C6-C8-cycloalkyl,

alkoxy: C1-C4-alkoxy,

long-chain alkoxy: linear and branched C5-C10 alkoxy, preferably linear C6-C8 alkoxy

alkylene: selected from the group consisting of: methylene; 1,2-ethylene; 1,3-propylene; butan-2-ol-1,4-diyl; 1,4-butylene; cyclohexane-1,1-diyl; cyclohexan-1,2-diyl; cyclohexan-1,4-diyl; cyclopentane-1,1-diyl; and cyclopentan-1,2-diyl,

aryl: selected from group consisting of: phenyl; biphenyl; naphthalenyl; anthracenyl; and phenanthrenyl,

arylene: selected from the group consisting of: 1,2-phenylene; 1,3-phenylene; 1,4-phenylene; 1,2-naphtalenylene; 1,4-naphtalenylene; 2,3-naphtalenylene and 1-hydroxy-2,6-phenylene,

heteroaryl: selected from the group consisting of:

pyridinyl; pyrimidinyl; quinolinyl; pyrazolyl; triazolyl; isoquinolinyl; imidazolyl; and oxazolidinyl, wherein the heteroaryl may be connected to the compound via any atom in the ring of the selected heteroaryl, heteroarylene: selected from the group consisting of: pyridin 2,3-diyl; pyridin-2,4-diyl; pyridin-2,6-diyl; pyridin-3,5-diyl; quinolin-2,3-diyl; quinolin-2,4-diyl; isoquinolin-1,3-diyl; isoquinolin-1,4-diyl; pyrazol-3,5-diyl; and imidazole-2,4-diyl,

heterocycloalkyl: selected from the group consisting of:

pyrrolidinyl; morpholinyl; piperidinyl; piperidinyl; 1,4 piperazinyl; tetrahydrofuranyl; 1,4,7-triazacyclononanyl; 1,4,8,11-tetraazacyclotetradecanyl; 1,4,7,10,13-pentaazacyclopentadecanyl; 1,4,7,10-tetraazacyclododecanyl; and piperazinyl, wherein the heterocycloalkyl may be connected to the compound via any atom in the ring of the selected heterocycloalkyl, heterocycloalkylene: selected from the group consisting of:

piperidin-2,6-ylene; piperidin-4,4-ylidene; 1,4-piperazin-1,4-ylene; 1,4-piperazin-2,3-ylene; 1,4-piperazin-2,6-ylene; tetrahydrothiophen-2,5-ylene; tetrahydrothiophen-3,4-ylene; tetrahydrofuran-2,5-ylene; tetrahydrofuran-3,4-ylene; pyrrolidin-2,5-ylene; pyrrolidin-2,2-ylidene; 1,4,7-triazacyclonon-1,4-ylene; 1,4,7-triazacyclonon-2,3-ylene; 1,4,7-triazacyclonon-2,2-ylidene; 1,4,8,11-tetraazacyclotetradec-1,4-ylene; 1,4,8,11-tetraazacyclotetradec-1,8-ylene; 1,4,8,11-tetraazacyclotetradec-2,3-ylene; 1,4,8,11-tetraazacyclotetradec-2,2-ylidene; 1,4,7,10-tetraazacyclododec-1,4-ylene; 1,4,7,10-tetraazacyclododec-1,7-ylene; 1,4,7,10-tetraazacyclododec-2,3-ylene; 1,4,7,10-tetraazacyclododec-2,2-ylidene; 1,4,7,10,13-pentaazacyclopentadec-1,4-ylene; 1,4,7,10,13-pentaazacyclopentadec-1,7-ylene; 1,4-diaza-7-thia-cyclonon-1,4 ylene; 1,4-diaza-7-thia-cyclonon-2,3-ylene; 1,4-diaza-7-thia cyclonon-2,2-ylidene; 1,4-diaza-7-oxa-cyclonon-1,4-ylene; 1,4 diaza-7-oxa-cyclonon-2,3-ylene; 1,4-diaza-7-oxa-cyclonon-2,2-ylidene; 1,4-dioxan-2,6-ylene; 1,4-dioxan-2,2-ylidene; tetrahydropyran-2,6-ylene; tetrahydropyran-2,5-ylene; and tetrahydropyran-2,2-ylidene, a —C1-C6-alkyl-heterocycloalkyl, wherein the heterocycloalkyl of the —C1-C6-heterocycloalkyl is selected from the group consisting of: piperidinyl; 1,4-piperazinyl; tetrahydrofuranyl; 1,4,7-triazacyclononanyl; 1,4,8,11-tetraazacyclotetradecanyl; 1,4,7,10,13-pentaazacyclopentadecanyl; 1,4,7,10-tetraazacyclododecanyl; and pyrrolidinyl, wherein the heterocycloalkyl may be connected to the —C1-C6-alkyl via any atom in the ring of the selected heterocycloalkyl,

amine: the group —N(R)2, wherein each R is independently selected from: hydrogen; C1-C6-alkyl; and benzyl,

halogen: selected from the group consisting of: F and Cl,

sulphonate: the group —S(O)2OR, wherein R is selected from: hydrogen; C1-C6-alkyl; Na; K; Mg; and Ca,

sulphate: the group —OS(O)2OR, wherein R is selected from: hydrogen; C1-C6-alkyl; Na; K; Mg; and Ca,

sulphone: the group —S(O)2R, wherein R is selected from: hydrogen; C1-C6-alkyl; benzyl and amine selected from the group: —NR′2, wherein each R′ is independently selected from:

hydrogen; C1-C6-alkyl; and benzyl,

carboxylate derivative: the group —C(O)OR, wherein R is selected from hydrogen; Na; K; Mg; Ca; C1-C6-alkyl; and benzyl,

carbonyl derivative: the group: —C(O)R, wherein R is selected from: hydrogen; C1-C6-alkyl; benzyl and amine selected from the group: —NR′2, wherein each R′ is independently selected from: hydrogen; C1-C6-alkyl; and benzyl,

phosphonate: the group —P(O)(OR)2, wherein each R is independently selected from: hydrogen; C1-C6-alkyl; benzyl; Na; K; Mg; and Ca,

phosphate: the group —OP(O)(OR)2, wherein each R is independently selected from: hydrogen; C1-C6-alkyl; benzyl; Na; K; Mg; and Ca,

phosphine: the group —P(R)2, wherein each R is independently selected from: hydrogen; C1-C6-alkyl; and benzyl,

phosphine oxide: the group —P(O)R2, wherein R is independently selected from: hydrogen; C1-C6-alkyl; benzyl and amine selected from the group: —NR′2, wherein each R′ is independently selected from: hydrogen; C1-C6-alkyl; and benzyl.

polyether: chosen from the group comprising —(O—CH₂—CH(R))ₙ—OH and —(O—CH₂—CH(R))ₙ—H whereby R is independently selected from: hydrogen, methyl, halogen and n is from 5 to 50, preferably 10 to 25.

M, Mₙ (n being an integer): Metals (either charged or uncharged), whereby two Metals Mₙ and Mₘ are independently selected from each other unless otherwise indicated.

These compounds have proven in practice to be suitable within the present invention.

According to a preferred embodiment of the present invention, the substrate material comprises a material according to structure II:

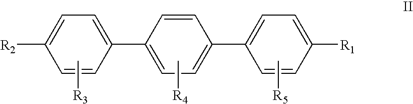

wherein R1, R2, R3, R4 and/or R5 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

According to a preferred embodiment of the present invention, the substrate material comprises a material chosen from the group comprising the structures III to VI:

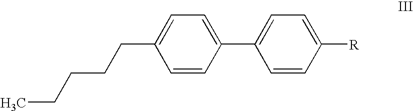

whereby R is chosen out of the group comprising halogens and pseudohalogens

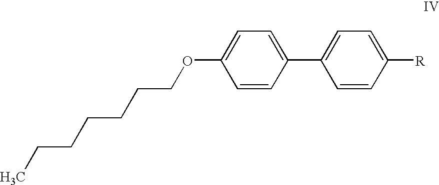

whereby R is chosen out of the group comprising halogens and pseudohalogens

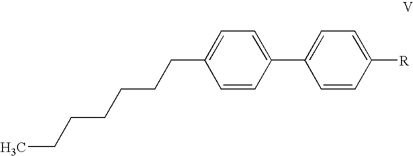

whereby R is chosen out of the group comprising halogens and pseudohalogens

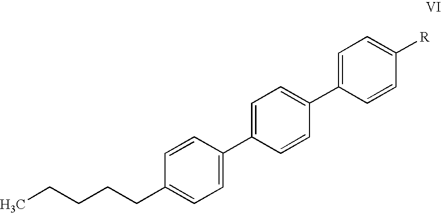

whereby R is chosen out of the group comprising halogens and pseudohalogens

or mixtures thereof. Preferably, the substrate material comprises a mixture of the compounds III to VI.

A preferred mixture of these materials is commercially available under the name of E7 (Merck KGaA, Frankfurter Str. 250, D-64293 Darmstadt, Germany), with all groups R being R=—CN. It is nematic at room temperature and has its nematic to isotropic transition at 58° C. Manipulating its phase transition can easily be done by blending it with other materials. For instance the material that is denoted as structure III has a nematic to isotropic transition of 35.5° C. Just increasing the amount of this compound decreases the transition almost linearly. If on the other hand higher temperatures are needed the component III should be added in a higher quantity.

This blend of so-called cyanobiphenyls is suited for analytes with a medium polarity. In case the analyte consist of molecules of low polarity, the group R is preferably chosen to be halogen, more preferably a fluoro group.

According to a preferred embodiment of the present invention, the substrate material comprises a material according to structure VII:

R₁-R₃-R₂ VII

wherein R1, and/or R2 are independently selected out of a group comprising cycloalkyl, aryl, arylene, halogenaryl, heteroaryl, heteroarylene, heterocycloalkylene, heterocycloalkyl, halogenheteroaryl, ketoaryl, halogenketoaryl, ketoheteroaryl either unsubstituted or substituted with one or more substituents selected out of the group comprising hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether;

and R3 is chosen out of the group comprising carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

According to a preferred embodiment of the present invention, R3 is chosen out of the group comprising ethylene, ethyl, alkinyl, ester, thioester, azo, azoxy, imino, butyl, 2-butylen, cyclohexyl, 2-cyclohexylen.

According to a preferred embodiment of the present invention, the substrate material comprises a material according to structure VIII to XIII:

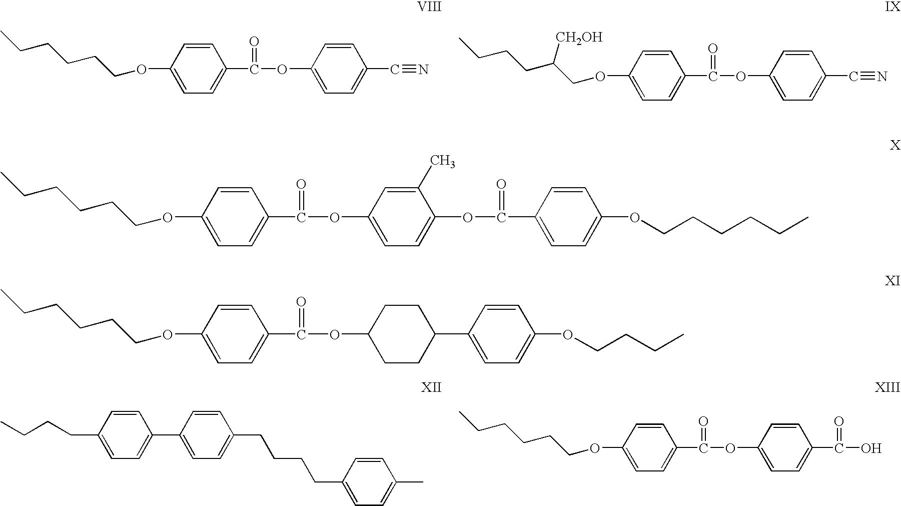

or mixtures thereof.

According to a preferred embodiment of the present invention, the substrate material comprises a polymeric liquid crystal material. These materials have shown to be suitable within the present invention.

According to a preferred embodiment of the present invention, the substrate material comprises a polymeric material selected out of the group polyacrylate, a polymethacrylate, a polyether, a polyester, a polypeptide or a polysiloxane or mixtures thereof, whereby liquid crystal molecules and/or structural moieties are attached as side groups to the polymer main chain.

According to a preferred embodiment of the present invention, the substrate material is a liquid crystal material, whereby the liquid crystal side chain moieties comprise at least one of the following structures XIV, XV and XVI:

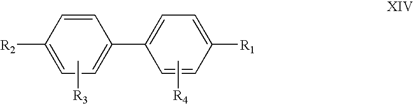

wherein R1, R2, R3 and/or R4 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

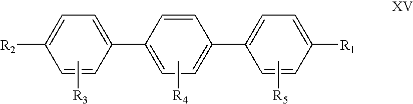

wherein R1, R2, R3, R4 and/or R5 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

R₁-R₃-R₂ XVI

wherein R1, and/or R2 are independently selected out of a group comprising cycloalkyl, aryl, arylene, halogenaryl, heteroaryl, heteroarylene, heterocycloalkylene, heterocycloalkyl, halogenheteroaryl, ketoaryl, halogenketoaryl, ketoheteroaryl either unsubstituted or substituted with one or more substituents selected out of the group comprising hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether;

and R3 is chosen out of the group comprising carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

The term “whereby the liquid crystal side chain moieties comprise the following structures” means especially that the structural moieties are present somewhere in the liquid crystal material.

According to a preferred embodiment, the percentage of side chains of the polymeric liquid crystal material, on which liquid crystal molecules and/or structural moieties are attached to is ≧0 and ≦100%, preferably ≧5 and ≦95%, more preferably ≧20 and ≦93% and most preferred ≧50 and ≦90%.

According to a preferred embodiment, the substrate material comprises a liquid crystal material is a partial-crosslinked polymeric material comprising the following structural moieties XVII and XVIII,

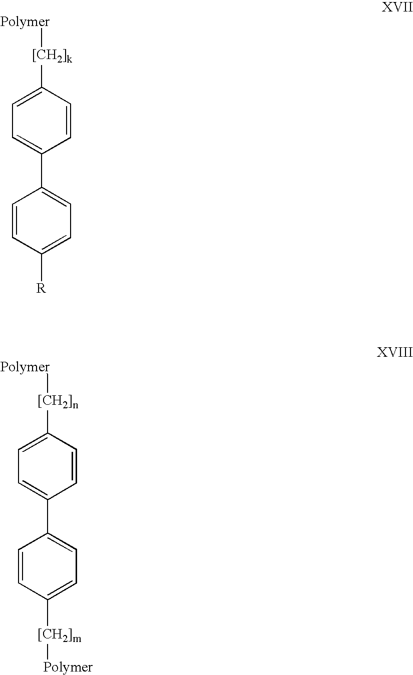

whereby R is selected from the group comprising halogens and pseudohalogens; and

k, n and m are independently chosen from each other integers from ≧2 to ≦18, preferably ≧3 to ≦12, more preferred ≧4 to ≦8.

According to a preferred embodiment of the present invention, the substrate material comprises a liquid crystal material is a partial-crosslinked polyether material comprising the following structural moieties XVII and XVIII as described, whereby the ratio of moieties according to structure XVII to moieties according to structure XVIII is from ≧2:1 to ≦2000:1, preferably ≧4:1 to ≦1000:1, more preferred ≧10:1 to ≦500:1.

According to a preferred embodiment of the present invention, the substrate material comprises a liquid crystalline gel material. These materials have shown to be best suitable according to some applications within the present invention.

In the sense of the present invention, a “liquid crystalline gel material” means and/or includes especially a mixture of a small molecular liquid crystal material with a molecular weight of <1500 Da and a polymeric liquid crystal material.

Preferably the ratio (wt:wt) of small molecular liquid crystal material to polymeric liquid crystal material is ≧11:1 to ≦200:1, more preferred ≧2:1 to ≦100:1 and most preferred ≧210:1 to ≦50:1.

Preferably the small molecular liquid crystal material is chosen from the structures I to XIII as described above and/or the polymeric liquid crystal material is chosen from the polymeric liquid crystal materials as described above.

The object of the present invention is furthermore solved by a method of influencing the flow of a sample or parts thereof in or with a substrate material according to the present invention, whereby the method comprises the step of causing a phase transition in at least on desired area of the substrate material According to a preferred embodiment of the present invention, the phase transition is caused by a change of temperature.

The object of the present invention is furthermore solved by a device comprising a substrate material according to the present invention, whereby the device is equipped with heating means which allow to cause a change of temperature at least one desired area of the substrate material.

According to a preferred embodiment of the present invention, the heating means are electrically controllable heating means, preferably indium tin oxide heating elements.

According to a preferred embodiment of the present invention, the device is an active or passive matrix type device for the controlled local heating of the substrate material. Such a device can e.g. be realized by electrically connecting each heating element or at least two electrodes associated or attributed to a heating element via at least one active component (in case of active matrix) to one of a plurality of row selection lines and/or to one of a plurality of column selectionsignal lines. The active matrix principle is realized by connecting at least one of the electrodes (first or second electrode attributed to each heating element) to the row selectionselection lines and/or the column selectionsignal lines via an active electrical or electronic component. Such active components include especially transistors like switch transistors (FET-transistors (field effect transistors) and/or bipolar transistors).

A substrate material, a method and/or device according to the present invention may be of use in a broad variety of systems and/or applications, amongst them one or more of the following:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
nano- and micro-fluidic devices
fluid pumping devices
drug release and drug delivery systems (in particular transdermal and implantable drug delivery devices).

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

Additional details, features, characteristics and advantages of the object of the invention are disclosed in the dependent claims, the figures and the following description of the respective figures and examples, which—in an exemplary fashion—show several preferred embodiments of a substrate material as well as a device according to the invention.

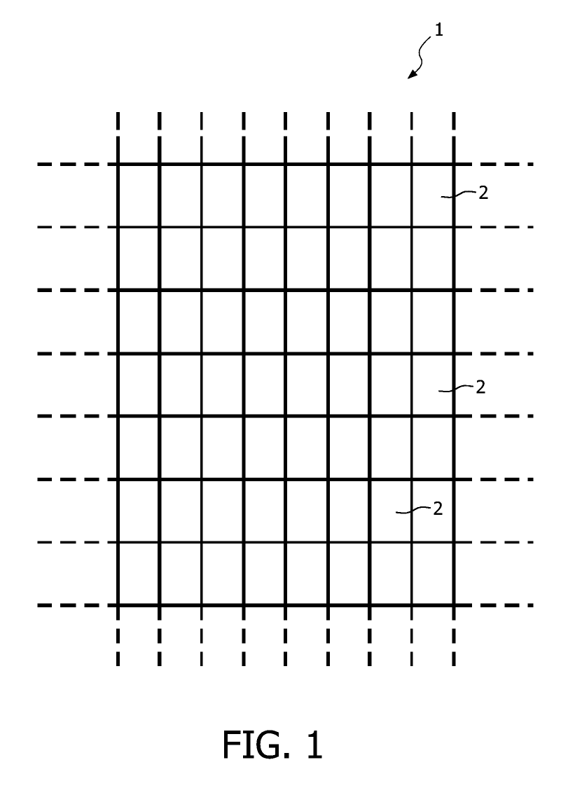

FIG. 1 shows a very schematic cross-sectional cut-out view of a substrate material according to a first embodiment of the present invention;

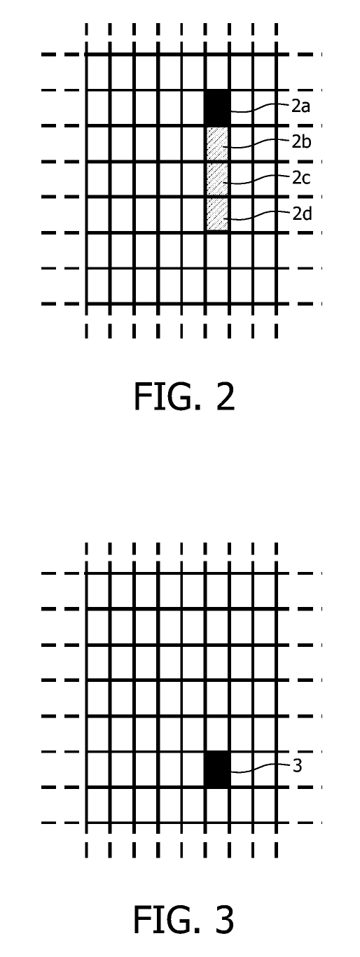

FIG. 2 shows the substrate material of FIG. 1 after addition of a sample and prior to the inducement of phase transition in certain selected areas in the substrate material;

FIG. 3 shows the substrate material of FIG. 2 after several phase transition in selected areas in the substrate material;

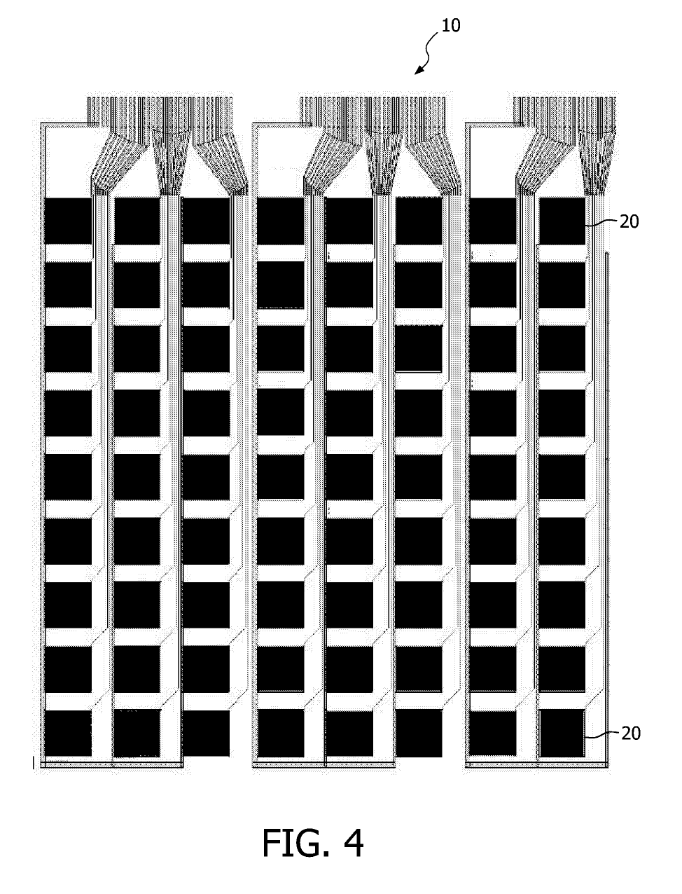

FIG. 4 shows a wiring pattern of heating means for a device according to a second embodiment of the present invention;

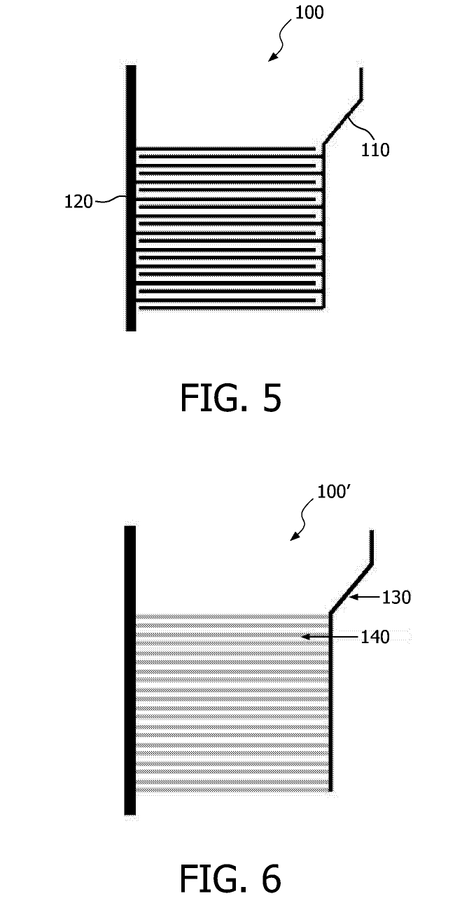

FIG. 5 shows a detailed view of a heating means of FIG. 4;

FIG. 6 shows a detailed view of an alternative heating means according to a third embodiment of the present invention; and

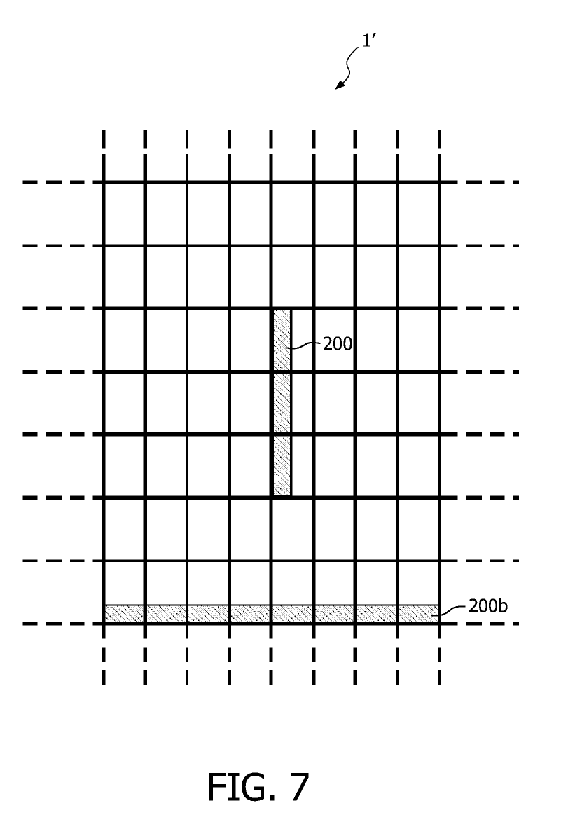

FIG. 7 shows a very schematic cross-sectional view of a substrate material according to a fourth embodiment of the present invention.

FIG. 1 shows a very schematic cross-sectional cut-out view of a substrate material according to a first embodiment of the present invention. The substrate material 1 consists out of a plurality of cells (some of which have been arbitrarily chosen and are referred to as numeral 2), which are usually somewhat square or rectangular in shape. By means, e.g. heating means as will described later on, a phase transition can be induced in each of the cells 2 essentially separately or independently. It should be noted that in the embodiment, all cells are more or less equal and therefore the numeral 2 refers for three arbitrarily chosen cells.

FIG. 2 shows the substrate material of FIG. 1 after addition of a sample and prior to the inducement of phase transition in certain selected areas in the substrate material. In this purely exemplarily embodiment, the sample has been added to cell 2a. After the addition (and an optional drying step) phase transitions are conducted in cells 2b, 2c and 2d, thus causing the sample or parts thereof to flow from cell 2a to 2d. It should be noted, that according to the chosen application, only parts of the sample e.g. macroparticles may be caused to flow within the substrate material 1 whereas the rest of the sample will remain in cell 2a.

According to an alternative and also preferred embodiment of the present invention, the substrate material is adapted in that way that a size-selective or size-dependent flow of macroparticles in the fluid in and/or with the substrate material is influenced and/or caused by phase transitions in selected areas of the substrate material. In FIG. 2 this would mean that smaller particles would e.g. be caused to flow to cell 2d, whereas larger particles would e.g. be caused to flow to cell 2b and medium-sized particles to cell 2c.

FIG. 3 shows the substrate material of FIG. 2 after several phase transition in selected areas in the substrate material. The sample (or parts thereof as described below) will then concentrate in a cell of the substrate material different from the cell where the sample was originally added to, e.g. cell 3. By further phase transition to selected areas of the substrate material, the sample (or parts thereof) may be shifted all over the substrate material 1 as desired.

It should be noted that (although this is not shown in the figs.) the cells are usually equipped with binding substances selective for certain analytes in the sample or the device, in which the substrate material is located in, is provided with further means which contain these binding substances (e.g. in the form that a further layer of material is provided, which contains such binding substances). When the sample comes across these binding substances, the corresponding analytes will be bound to the binding substances for analysis. It is obvious that by the possibility to “move” or “shift” the sample around the substrate material, the selectivity and resolution of the analysis device is greatly enhanced and the minimal required amount of fluid is decreased to a great extend as compared with the prior art.

FIG. 4 shows a wiring pattern 10 of heating means 20 for a device according to a second embodiment of the present invention, FIG. 5 shows a detailed view of a heating means of FIG. 4. In this embodiment the heating means 20 is embedded in a bottom glass plate and is equipped with a structured ITO resistor element such that interdigitated electrodes are applied over which a voltage can be applied. As shown in FIG. 4, the electrodes 110 and 120 are wound around each other in a serpentine-like structure, thus the current between the ITO electrodes has to pass in this case the liquid crystal film (not shown in the figs). Because of the resistance, the liquid crystal mixture heats up and undergoes a phase transition to the isotropic phase. In some applications it is possible to tune the resistivity by the addition of (semi)conductive molecular entities to the substrate material The bottom glass plate is adhered to a top glass leaving a spacing of 20 μm. The top plate is provided with small holes through which analytes and reagents can be added and eventually withdrawn for the micro-reactor. To improve on the conductivity enabling current-driven heating conductive materials can be added in small quantities, for instance ionic liquid crystal materials.

In the embodiment of FIGS. 4 and 5, the electrodes 110 and 120 have a relatively small width of e.g. 10 μm and the distance between the electrodes is chosen to be relatively small, e.g. 5 μm, whereas the total surface of the element that is addressed may be quite larger, e.g. 1000×1000 μm.

FIG. 6 shows a detailed view of an alternative heating means according to a third embodiment of the present invention. In this embodiment electrodes are directly connected via resistor material, e.g. ITO, thin-film copper, carbon-filled polymers, etc. Therefore an area with a high conductivity 140 and resistor area with a low conductivity 130 results. Due to the resistance of the low conductivity area, also heat is generated. Instead of having a line pattern of resistor material 130 also a continuous sheet resistor may be present according to a further preferred embodiment of the present invention (not shown in the figs.).

FIG. 7 shows a very schematic cross-sectional view of a substrate material 1′ according to a fourth embodiment of the present invention. This embodiment differs from that in the FIGS. 1 to 3 in that certain areas 200 are present, which are “pre shaped” and serve as a nucleus for phase transitions in the substrate material, i.e. the substrate material is permanently modified in a way that nucleation of one of the phases occurs preferably at those sides. E.g. in case that a phase transition from a nematic to a smectic state should be realized in the substrate material by a temperature decrease, these areas 200 may comprise a material which employs features of the smectic state e.g. in that the order is similar. In this example the material in areas 200 could be a smectic phase, which is fixed (by e.g. photo-polymerization), i.e. it does not switch to the nematic nor isotropic phase if heated above the phase transition temperature(s). When causing a phase transition, a preferred direction in which these phase transitions occur is introduced by these areas 200. This allows a more directed flow of the sample or parts thereof in and/or with the substrate material.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The scope of the invention is defined in the following claims and the equivalents thereto. Furthermore, reference signs used in the description and claims do not limit the scope of the invention as claimed.

1. A substrate material for use in a device analyzing one or more samples for the presence, amount or identity of one or more analytes in the samples, whereby the substrate material is adapted in that way that a flow of the sample or parts thereof in and/or with the substrate material is influenced and/or caused by phase transitions in selected areas of the substrate material.

2. A substrate material according to claim 1, whereby the phase transitions in the substrate material include reversible phase transitions.

3. A substrate material according to claim 1, whereby the phase transitions in the substrate material include temperature-inducible phase transitions.

4. A substrate material according to claim 1, whereby the substrate material includes a liquid crystal material.

5. A substrate material according to claim 1, whereby the substrate material comprises a material chosen from the group comprising the structures I, II and III 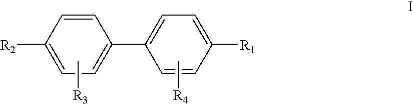
wherein R1, R2, R3 and/or R4 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether, nitrile. 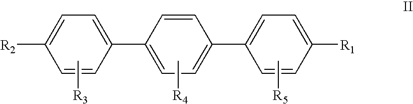
wherein R1, R2, R3, R4 and/or R5 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.
R₁-R₃-R₂ III
wherein R1, and/or R2 are independently selected out of a group comprising cycloalkyl, aryl, arylene, halogenaryl, heteroaryl, heteroarylene, heterocycloalkylene, heterocycloalkyl, halogenheteroaryl, ketoaryl, halogenketoaryl, ketoheteroaryl either unsubstituted or substituted with one or more substituents selected out of the group comprising hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether;
and R3 is chosen out of the group comprising carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

6. A method of influencing the flow of a sample or parts thereof in or with a substrate material according to the present invention, whereby the method comprises the step of causing a phase transition in at least one desired area of the substrate material.

7. A method according to claim 6, whereby the phase transition is caused by a change of temperature.

8. A device comprising a substrate material according to claim 1, whereby the device is equipped with heating means which allow to cause a change of temperature at least one desired area of the substrate material.

9. A device according to claim 8, whereby the heating means are electrically controllable heating means, preferably indium tin oxide heating elements.

10. A system incorporating a substrate material for use in a device analyzing one or more samples for the presence, amount or identity of one or more analytes in the samples, whereby the substrate material is adapted in that way that a flow of the sample or parts thereof in and/or with the substrate material is influenced and/or caused by phase transitions in selected areas of the substrate material, adapted to conduct the method of claim 6 and being used in one or more of the following applications:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
nano- and micro-fluidic devices
fluid pumping devices
drug release and drug delivery systems (in particular transdermal and implantable drug delivery devices).
